(19) United States
(12) Patent Application Publication
(10) Pub. No.: US 2015/0158021 A1
(43) Pub. Date: Jun. 11, 2015

(54) PROCESS FOR THE IMMOBILIZATION OF CATALYSTS ON TEXTILE MATERIALS, THE OBTAINED TEXTILE MATERIALS AND THE USE OF SAID MATERIALS

(71) Applicant: STUDIENGESELLSCHAFT KOHLE MBH, Mülheim an der Ruhr (DE)

(72) Inventors: Ji-Wong Lee, Mulheim an der Ruhr (DE); Benjamin List, Mulheim an der Ruhr (DE); Klaus Opwis, Kevelaer (DE); Thomas Mayer-Gall, Kamp-Lintfort (DE); Jochen Stefan Gutmann, Krefeld (DE)

(73) Assignee: STUDIENGESELLSCHAFT KOHLE MBH, Mülheim (DE)

(21) Appl. No.: 14/403,690

(22) PCT Filed: May 28, 2013
(86) PCT No.: PCT/EP2013/060991

§ 371 Date: Nov. 25, 2014

(30) Foreign Application Priority Data
May 31, 2012 (EP) 12170144.5

(51) Int. Cl. B01J 31/12; C07C 47/54; C07C 67/29; C07C 49/173; B01J 37/34; C07C 67/08

(52) U.S. Cl. CPC B01J 31/123; B01J 37/345; C07C 67/08; C07C 67/29; C07C 49/173; C07C 47/54; B01J 2231/482; B01J 2531/002

(57) ABSTRACT

The present invention relates to methods for the preparation of solid-supported heterogeneous organic catalyst covalently bound to textile materials, preferably via photochemical immobilization. More specifically, the present invention relates to the organocatalysis and recognition process by using the textile-supported chiral molecules.

This application is a 371 of International Application No. PCT/EP2013/060991, filed May 28, 2013, which, in turn, claims priority of European Patent Application No. EP 12170144.5, filed May 31, 2012, the entire disclosures of which patent applications are hereby incorporated by reference herein.

The present invention relates to processes for the preparation of solid-supported organic catalysts, particularly on textile materials via covalent bonding, in particular via photochemical immobilization. Furthermore, the present invention relates to processes making use of the textile-supported catalysts, in particular in the synthesis of chiral molecules using organocatalysis.

Catalysts lower the activation energy of chemical reactions compared to the non-catalyzed reaction. This reduces the energy requirement of a reaction drastically, and enables the environmentally friendly and economical chemical production of a variety of valuable compounds. Practically, most of well-known chemical processes employ heterogeneous catalysts for easy separation of the product and convenient recycling of the catalyst. Also, heterogeneous catalysts can easily be applied to continuous flow process. Moreover, it is desirable to establish methodology for the preparation of heterogeneous variants of homogeneous catalysts via simple and practical modification procedures.

Heterogeneous catalysis can be divided into two groups:
Immobilized metals and metal oxides with large and active surface area, for example, a catalytic converter which converts toxic chemicals to less toxic ones for most of the vehicles, the Haber process for ammonia synthesis and the Fischer-Tropsch process for the conversion of carbon monoxide and hydrogen into liquid hydrocarbons as an example of a heterogeneous transition metal catalyzed process.

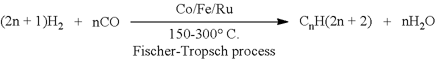

Immobilized organic molecules with an active site for:
   1) transition metal catalysis as a ligand
   2) biocatalysis using high-molecular-weight enzymes, and
   3) organocatalysis, especially using chiral organocatalysts for asymmetric syntheses.

During the last dozen years, organocatalysis emerged as a major field of asymmetric synthesis complementing biocatalysis and transition metal catalysis. Its versatility has been established in numerous organic transformations with unprecedented activity and high selectivity. By employing small organic molecules, the reactions could be catalyzed via covalent activation, hydrogen bonding interaction and ionic interactions. To achieve reasonable reaction outcome with high selectivity, it is often required to use a fairly large catalyst loading which may hamper an industrial application of organocatalysis.

To overcome above mentioned obstacles and to realize the possibility of recycling the organic catalyst, a number of strategies have been explored for preparing heterogeneous organocatalysts via covalent and non-covalent immobilization. For example, the Jacobsen group utilized Merrifield's resin for the preparation of a library of thiourea catalysts for asymmetric Strecker synthesis. Extensive studies were conducted for immobilization of secondary amine catalysts via co-polymerization, click chemistry, ionic liquid formation. Also, functionalization of alkene functional group can give a facile access for heterogeneous organocatalyst. For example, hydrosilylation of cinchona alkaloid catalyzed by platinum catalyst (i.e. Speier's and Karstedt's) can generate silylated compounds, which can be grafted onto a heterogeneous surface.

All the described methods have significant drawbacks. First of all, the methods, using polystyrene based resin (i.e. Merrifield resin, Wang resin, JandaJel™) for immobilization, require complicated modifications of the monomeric catalyst or the resin itself to install an appropriate functional group. In this process, unreacted functional groups remain on the surface and will affect or interrupt the catalysis. Moreover, additional functional groups on the catalyst may alter the activity and selectivity of the heterogenized catalyst versus its homogenous counterpart. Furthermore, such modification processes often require multiple organic transformations, which ultimately prevent industrial applications.

Recently, co-polymerization of monomeric catalysts and self-supported catalysts (“bottom-up” strategy) to produce heterogeneous organocatalysts are emerging as an alternative pathway. Cowley's group has reported the catalysis with a heterogeneous imidazolium salt as a carbene precursor. This orthogonally positioned heterogenous carbene catalyst was used for three cycles without great loss of reactivity. However, this methodology also requires complicated pre-modification of the catalyst monomer. Oxidative polymerization of thiophene functional group can directly generate porous heterogeneous organocatalyst without sophisticated modification of catalyst monomer. However, since the thiophene substituent can alter the activity and selectivity of the catalysis, stereochemical outcome of the heterogeneous catalysis was inferior to homogenous catalysis with bulky substituents.

To solve this limitation, it is a great challenge to find versatile solid material which is robust and abundant for large scale application. As already mentioned, the advantage of heterogeneous catalysis is that no separation of the catalyst is required and that the catalyst can be recycled without sophisticated purification. In accordance with this ultimate goal, the morphology of the heterogeneous material has to be easy to handle for various applications. To address this problem, various catalytic resins were prepared with the bead form not displaying powder morphology. However, this requires additional engineering technology for their preparation. Also, for high performance of the heterogeneous catalyst, it is highly desirable to obtain high mass transfer efficiency to the active sites and prevent catalyst leach-out during the catalysis, which requires a permanent chemical connection between catalyst and solid support. Also, the total porosity and the pore distribution have an important role. These parameters directly influence the catalyst loading capacity of the material, the material conversion to the corresponding immobilized catalyst, and the separation ability of the overall system of the reaction matrix.

Concerning all these parameters, heterogeneous catalysts that are immobilized in various ways are relatively expensive and not applicable for large scale production. Therefore, it is highly desirable to develop an efficient immobilization technique, which can be applied to the industrial use of asymmetric organocatalysts.

The inventors found out that a substrate-specific immobilization method can provide permanently immobilized catalysts on polymeric materials such as textile surfaces via a photo-induced cross-linking reaction. To provide a general solution for the described problems, the presented invention is related to the facile immobilization of organocatalysis on a suitable textile material via a covalent bonding, preferably via simple one-step photochemical process. After covalent immobilization, heterogeneous organocatalysts can not be leached-out during the reaction and/or recycling process. Leaching-out problem is often observed in heterogeneous organometallic catalysis, which will eventually contaminate the reaction product with transition metal impurities.

The solution of the problem underlying the invention is achieved by providing a general method for an immobilization of organic catalysts on the polymeric carrier materials, specifically, textile materials. The immobilization process is conducted by covalently bonding the catalyst to the catalyst carrier, preferably by using UV light irradiation of a solid material, specifically, textile materials and subsequent quenching with appropriate organic molecules, which are immobilized on the solid surface covalently.

The invention is therefore directed to a process for immobilization of at least one organic catalyst on a polymeric support, wherein said organic catalyst is permanently bonded to the polymeric support by a covalent bonding between at least one functional group on either of the organic catalyst and the polymeric support. The covalent bonding can be achieved via bonding in chemical reactions forming C—C—, C—N—, or C—O-bonds.

The covalent bonding is generally a ionically induced or radically induced reaction, and can be induced by a wet chemical process or a photochemical process.

The prepared textile-immobilized organic catalyst is a heterogeneous organic catalyst, which does not have any metal-centered catalytically active center and is preferably selected from any of cinchona alkaloid-based bifunctional catalysts, BINOL-based phosphoric acids, BINOL-based imidodiphosphoric acids, secondary- and primary amine catalysts, nitrogen-based nucleophilic catalysts, TEMPO as an organic oxidant, phase-transfer catalysts as well as imidodiphosphoric acids as disclosed in EP application No. 12150663.8 of the same applicant. The catalyst is particularly a chiral heterogeneous organic catalyst.

As indicated before for one embodiment of the invention, the present invention makes, amongst others, use of chiral imidodiphosphates and derivatives thereof acids as disclosed in EP application No. 12150663.8 of the same applicant and having the general formula (I), which have been described in EP12150663.8, as follows:

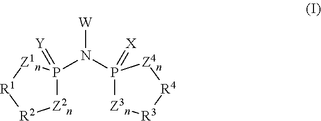

wherein:

X and Y may be, independently from each other, the same or different and represent O, S, Se and NR^{N},

Z¹ to Z⁴ may be, independently from each other, the same or different and represent O, S and NR^{N},

n stands for 0 or preferably 1,

W may be substituent being capable of forming a covalent or, preferably, a ionic bond with the imidodiphosphate moiety,

R¹ to R⁴ may be, independently from each other, the same or different and may be each an aliphatic, heteroaliphatic, aromatic or heteroaromatic group, each optionally being further substituted by one or more heterosubstituents, aliphatic, heteroaliphatic, aromatic or heteroaromatic groups whereby R¹ and R² are forming a ring system with Z¹ and Z² and R³ and R⁴ are forming a ring system with Z³ and Z⁴, respectively, and

R^{N} may be selected from hydrogen, C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl or heterosubstituents, including its tautomeric and ionic forms, and derivatives thereof.

In the following, it is to be understood that the above formula (I) comprises its tautomeric forms as represented by the formulae (Ia) or (Ib)

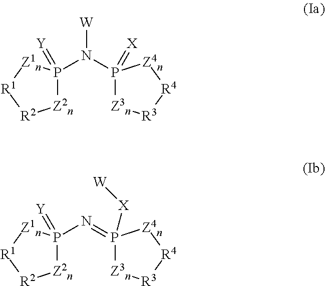

wherein X, Y, Z¹ to Z⁴, n, W, R¹ to R⁴ and R^{N} have the meaning as defined above. In the following, it is to be understood that any of the formulae (II), (III), (IV) and (V) below comprises its respective tautomeric forms as represented by formula (Ia) or formula (Ib).

In the present application, the expression “imidodiphosphates” is to be understood to comprise derivatives thereof, wherein one or more of the oxygen atoms of the imidodiphosphate moiety is replaced by S, Se, NR^{N} as defined above.

In the above formula (I) and the derived formulae below, it is to be understood that any tautomeric form of the inventive chiral imidodiphosphates as well as any charged form thereof including any anionic form is to be comprised by the representation of said formula. It is also to be understood that imidodiphosphates could possess inherent chirality even if all of the groups R¹ to R⁴ are achiral groups.

In the above formulae (I), R¹ to R⁴ may be selected each from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, or C₆ to C₂₀ aromatic hydrocarbon and partially arene-hydrogenated forms such as aryl, aryl-(C₁-C₆)-alkyl, heteroaryl-(C₁-C₆)-alkyl or heterosubstituents.

In the above formula (I), W is a substituent being capable of forming a covalent or ionic bond with the imidodiphosphate moiety such as hydrogen, —OH, halogen, a metal such as Li, Na, K, Rb, Cs, Be, Mg, Ca, Sr, Ba, Sc, Ti, V, Cr, Mn, Fe, Co, Ni, Cu, Zn, Y, Zr, Mo, Ru, Rh, Pd, Ag, W, Re, Os, Ir, Pt, Au, Al, Pb, La, Sm, Eu, Yb, U, or a cationic organic group as exemplified in Scheme 2 below, R^{w} or a substituted silicon such as —SiR^{I}R^{II}R^{III}, wherein R^{w}, R^{I}, R^{III} and R^{III} may be same or different and each stand for hydrogen, halogen, C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl or a heterosubstituent.

The expression “partially arene-hydrogenated forms thereof” is to be understood that in case that the aromatic structure comprises more than one aromatic cycle such as for naphthalene, at least one aromatic cycle, one aromatic cycle remaining, might be partially or fully hydrogenated.

The anionic form may be complemented by any cation for forming an ion pair.

In one embodiment of the above formulae (I), Z¹ to Z⁴ represent O, n is 1 and the other definitions are as given before for formula (I), as represented by formula (II):

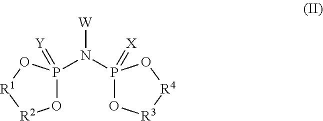

In such formulae (I) and (II), the moiety

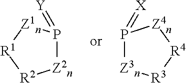

might be a five to ten-membered ring structure of (R¹, R², Z¹, Z² and —PY—) or (R³, R⁴, Z³, Z⁴ and —PX—), respectively.

In one embodiment of the compounds of formula (II), X and Y represent O and the other definitions are as given before for formulae (I), as represented by formula (III):

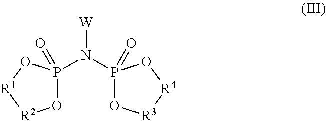

In such formula (III), at least one of (R¹ and R²) and (R³ and R⁴) may form a ring structure derived from a bridged aromatic structure such as biphenyl optionally substituted, BINOL, TADDOL, VAPOL, SPINOL, 1,1′-binaphthalene, 1,1′-bianthracene, 1,1-biphenanthrene, as well as the partially arene-hydrogenated forms such as 8H-BINOL, each of said rings systems optionally being substituted by one or more substituents selected from heterosubstituents, C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon such as aryl, aryl-(C₁-C₆)-alkyl, heteroaryl-(C₁-C₆)-alkyl, each hydrocarbon optionally being substituted by one or more heterosubstituents. In such formula (III), the ring structure formed by (R¹ and R²) or (R³ and R⁴) may be the same or different.

Examples of said compound having the formula (III) being generally usable are shown below:

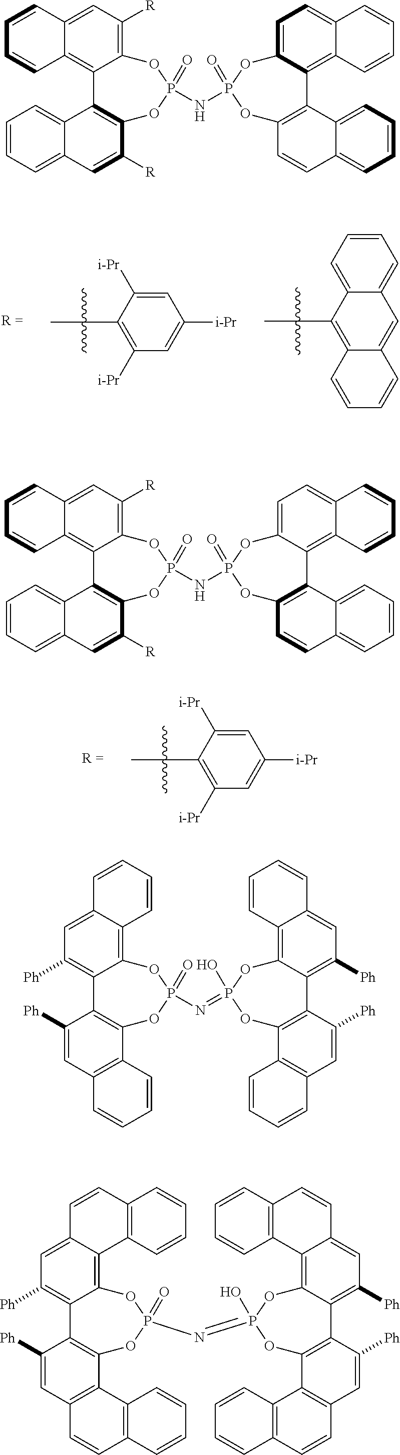

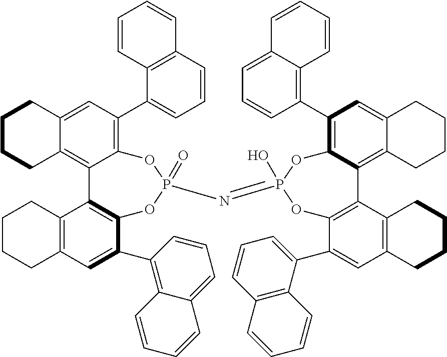

In a further embodiment, the compounds of formula (I) may be represented by formula (IV):

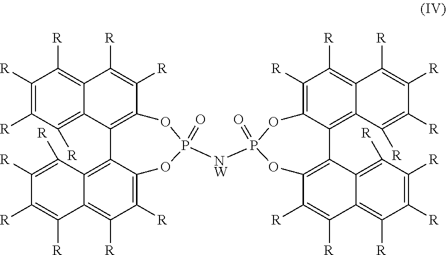

In said formula (IV), the substituent R may be the same or different on each position and may each stand for hydrogen, a heterosubstituent, C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl or a heterosubstituent.

In said formula (IV), W is defined as given before for formula (I).

The substituents on the ring structure proximal to the —Z—P— bond, such as the —O—P-bond, are preferably bulky groups and may be selected from the definitions for R^{N} or heterosubstituents.

In the inventive processes, the chiral imidodiphosphates having the general formula (II), (III) or (IV) are preferably used.

Basically, any chiral groups are possible as chiral groups for the inventive compounds. If the other group in each case is not chiral, the groups R¹ to R⁴ are any organic group which may be saturated or unsaturated, linear, cyclic or heterocyclic, aromatic and/or heteroaromatic.

Examples of said compound having the formula (IV) and prepared by the inventors are shown below:

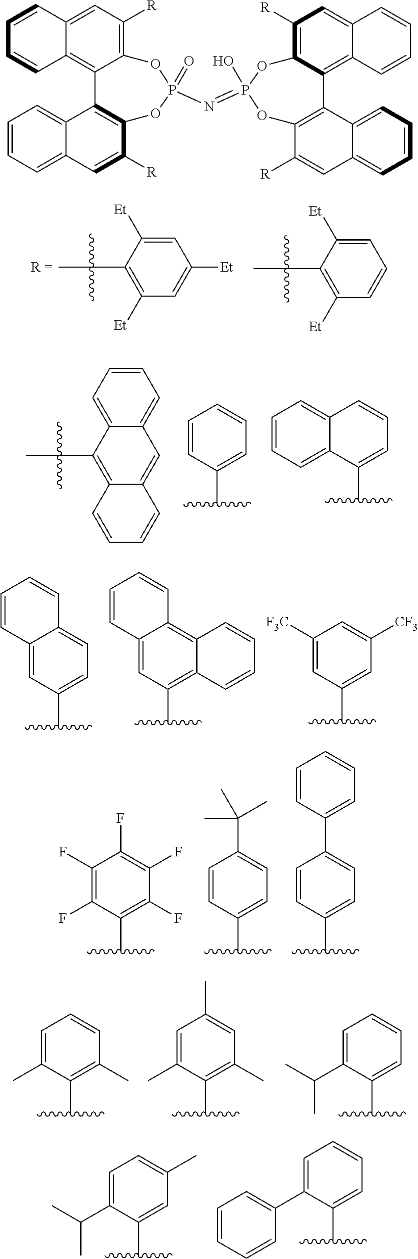

In organic synthesis, particularly in the synthesis of pharmaceutical active compounds, chiral compounds are frequently used as catalysts in order to obtain the desired product in a high enantiomeric purity or diastereomeric purity.

It has been found that the compounds according to the invention are well suited as catalysts for enantioselective synthesis. Here, they function as chiral Brønsted acids or the conjugated bases thereof as chiral anions in enantioselective catalyses directed by counterions.

The following definitions for the individual substituents/groups apply equally as follows.

A heterosubstituent as defined according to the invention can be selected from, ═O, OH, F, CI, Br, I, CN, NO₂, SO₃H, a monohalogenomethyl group, a dihalogenomethyl group, a trihalogenomethyl group, CF(CF3)₂, SF₅, amine bound through N atom, —O-alkyl (alkoxy), —O-aryl, —O—SiR^{S}₃, S—R^{S}, S(O)—R^{S}, S(O)₂—R^{S}, COOH, CO₂—R^{S}, amide, bound through C or N atom, formyl group, C(O)—R^{S}, COOM, where M may be a metal such as Na or K. R^{S}₃ may be, independently from each other, the same or different and may be each an aliphatic, heteroaliphatic, aromatic or heteroaromatic group, each optionally being further substituted by one or more heterosubstituents, aliphatic, heteroaliphatic, aromatic or heteroaromatic groups.

Aliphatic hydrocarbons including alkyl, alkenyl and alkinyl may comprise straight-chain, branched and cyclic hydrocarbons.

Heteroaliphatic is a hydrocarbon including alkyl, alkenyl and alkinyl which may comprise straight-chain, branched and cyclic hydrocarbons with one or more carbon atoms substituted with a heteroatom.

In more detail, C₁-C₂₀-Alkyl can be straight chain or branched and has 1, 2, 3, 4, 5, 6, 7, 8, 9, 10, 11, 12, 13, 14, 15, 16, 17, 18, 19 or 20 carbon atoms. Alkyl might be C₁-C₆-alkyl, in particular methyl, ethyl, propyl, isopropyl, butyl, isobutyl, sec-butyl or tert-butyl, likewise pentyl, 1-, 2- or 3-methylpropyl, 1,1-, 1,2- or 2,2-dimethylpropyl, 1-ethylpropyl, hexyl, 1-, 2, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl. Substituted alkyl groups are trifluoromethyl, pentafluoroethyl and 1,1,1-trifluoroethyl.

Cycloalkyl might be cyclopropyl, cyclobutyl, cyclopentyl, cyclohexyl or cycloheptyl. Alkenyl might be C₂-C₂₀ alkenyl. Alkinyl might be C₂-C₂₀ alkinyl.

Said unsaturated alkenyl- or alkinyl groups can be used for linking the inventive compounds to a carrier such as a polymer to serve for an immobilized catalyst.

Halogen is F, Cl, Br or I.

Alkoxy is preferably C₂-C₁₀ alkoxy such as methoxy, ethoxy, propoxy, tert-butoxy etc.

C₃-C₈-Heterocycloalkyl having one or more heteroatoms selected from among N, O and S is preferably 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl.

Optionally substituted means unsubstituted or monosubstituted, disubstituted, trisubstituted, tetrasubstituted, pentasubstituted, or even further substituted for each hydrogen on the hydrocarbon.

Aryl might be phenyl, naphthyl or biphenyl.

Arylalkyl might be benzyl.

Heteroaryl having one or more heteroatoms selected from among N, O and S is preferably 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, also preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-Indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, also preferably 1,3-benzodioxol-5-yl, 1,4-benzodioxan-6-yl, 2,1,3-benzothiadiazol-4- or -5-yl or 2,1,3-benzoxadiazol-5-yl.

In a preferred embodiment of the present invention as for example shown in formula (IV), at least one of R proximal to the —O—P— bond is not hydrogen and may be selected from among methyl, ethyl, isopropyl, cyclohexyl, cyclopentyl, phenyl, 2,4,6-triisopropylphenyl, 2,4,6-triethylphenyl, 2,6-diethylphenyl, 2,6-diethylphenyl, 2-isopropyllphenyl, 5-methyl-2-isopropyllphenyl, mesityl, 9-phenanthryl, 9-anthracenyl, ferrocenyl, N-(perfluorophenyl)acetamide, N-(4-chlorophenyl)acetamide, N-(naphthalen-1-yl)acetamide, N-benzhydrylacetamide, N-(2,6-diisopropylphenyl)acetamide, 1-anthracenyl, corannulene, porphyrin, 1-naphthyl, 2-naphthyl, 4-biphenyl, 3,5-(trifluoromethyl)phenyl, 2,6-dimethylphenyl, tert-butyl, tris-methylsilyl, tert-butydimethylsilyl, phenyldimethylsilyl, methyldiphenylsilyl, tris-mesitylsilyl, tris-phenylsilyl, 4-nitrophenyl and 2,6-methyl-4-butylphenyl, trifluoromethyl, unbranched (linear) and branched (C₁-C₁₂)-perfluoroalkyls, 3,4,5-trifluorophenyl, 1,3-bis(perfluoropropan-2-yl)phenyl, 1,3-bis(perfluorobutyl)phenyl and/or pentafluorophenyl and also chloride, iodide, fluoride, COOH, B(OH)₂, B(alkyl)₂, B(O-alkyl)₂, B(pinacol), BF₃X where X=Na or K, OTf. The other groups are preferably hydrogen.

The compounds according to the invention can be converted in process steps which are well known per se to those skilled in the art into organic salts, metal salts or metal complexes. In one possible embodiment, the imidodiphosphates are reacted with an appropriate metal salt, for example with the carbonate of the appropriate metal.

The polymeric support, a textile, is selected from the group of natural or synthetic textile materials or mixtures thereof, preferably selected from polyesters, polyamides, polyacrylates, polyolefins, cotton, rayon and wool.

Though the interaction is preferably induced by way of a photochemically induced process such as irradiation with UV light, it is also possible to induce said immobilization via a wet chemical process.

As preferred measure, the organic catalyst is immobilized on the polymeric support via a photochemical reaction between at least one functional crosslinking olefinic group on the organic catalyst with the polymeric support, either directly or via a linker molecule having at least two vinylic or allylic olefinic groups such as PETA or TAC.

The functional crosslinking olefinic group is advantageously a vinyl or allyl group attached to the organic catalyst, and the photochemical reaction is induced by irradiation with UV light, in particular with UV light of a wavelength in the range of 100 to 350 nm, preferably with a wavelength of 222 nm.

In one embodiment, the polymeric support is treated or impregnated with a solution of the organic catalyst having at least one functional crosslinking olefinic group in a photochemically inert organic solvent, the obtained impregnated polymeric support is irradiated with UV light and the obtained polymeric support having the organic catalyst covalently fixed thereon is recovered and optionally washed with an organic solvent. Photochemically inert organic solvent means that the solvent is capable of dissolving the used catalyst, but does not take part in the photochemical reaction or interaction between the catalyst moiety and the polymeric support. Leading to the catalyst immobilized on the polymeric support.

Thus, the invention provides a simple and rapid method for permanent immobilization of organic catalysts on polymeric textiles. Various catalysts can be immobilized and used several times without loss of activity. Particularly, textile materials provide a solid substrate by the good permeability for organic solvent and show excellent substrate conversion to the desired product with high selectivity. In addition, the flexibility of solid materials allows a practical use in various types of reactors without sophisticated packing or reaction set-ups.

Polymeric textile materials have been used since the pre-historic periods because of their high accessibility and numerous advantageous functionalities. One can use basically any textile materials, for example, those from vegetable, animal and insects. However, synthetic textile materials such as polyesters, polyamides, polyacrylates or polyolefins are much cheaper than styrene-based resins and highly durable under various conditions and easily accessible.

Moreover, due to the flexible textile structure it is highly applicable to diverse reactors with any geometry. In addition, textile fabrics and immobilized catalyst can be removed quickly and without leaving any residue from a reactor. The structure of low-bonded non-woven products ensures a large specific surface area and high permeability. Also textile materials can potentially render high catalyst loading and rapid mass transfer and thus it enables high turnover number and frequency. Finally, various textile materials with different chemical backbones render a variety of choices for immobilization process to produce comparable environment effect as homogeneous catalysis.

For the purpose of the present invention, various organocatalysts, which show high catalytic efficiency for valuable organic transformations, were immobilized on textile materials. This organocatalyst can be, for example, any of cinchona alkaloid-based bifunctional catalysts (A), BINOL-based phosphoric acids (B), BINOL-based imidodiphosphoric acids (C) including those, secondary- and primary amine catalysts (D), nitrogen-based nucleophilic catalysts (E), TEMPO as an organic oxidant (F) and phase-transfer catalysts (G) in FIG. 1 and those imidodiphosphoric acids as disclosed in EP application No. 12150663.8 as mentioned above. All the catalysts are preferably functionalized with an olefin functional group for the photochemical immobilization.

The invention relates to methods of preparation of organocatalyst-supported materials and the use of the same, in particular for catalysis in chemical reactions, more specifically, asymmetric organic reactions. All the methods make use of an organocatalyst having at least one olefin functional group for the photochemical immobilization on the textile carrier, preferably selected from any of polyesters, polyamides, polyacrylates or polyolefins or copolymers thereof. The immobilization can take place via a photochemical immobilization step or via a wet chemical immobilization step.

First, the inventive photochemical immobilization can generally proceed as follows: The organic catalyst functionalized with at least one carbon-carbon double bond is dissolved in a suitable solvent. A polymeric support material is wetted with the solution. Subsequently, the polymeric carrier material is irradiated with UV light. By the irradiation, the organic compound is permanently fixed to the support material.

For the photochemical immobilization, UV light has an appropriate wavelength that initiates the grafting reaction according to the invention, for example, to bind the olefin group to the textile materials, UV light with a wavelength ranging from 100 nm to 350 nm can be used. If it is necessary, to increase catalyst loading, catalytic activity and selectivity cross linker can be used. Such linker can provide two or more binding sites for one organocatalyst per binding site, respectively, each organocatalyst having at least one olefin functional group, so that more than one, at least two organocatalysts can be bonded to the carrier via said linker.

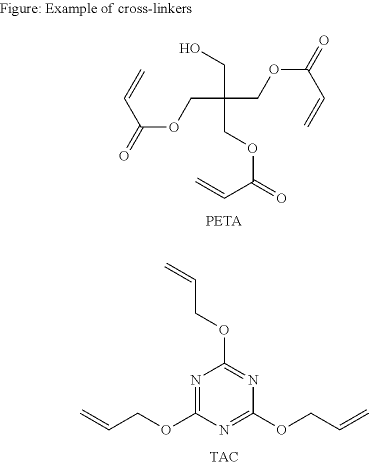 [SMILES: C=CC(=O)OCC(CO)(COC(=O)C=C)COC(=O)C=C ; C=CCOc1nc(OCC=C)nc(OCC=C)n1]

The invention is further illustrated by the following examples and attached drawings.

### BRIEF DESCRIPTION OF THE DRAWINGS

In said drawings,

FIG. 1 shows some illustrative embodiments of the catalysts which can be immobilized on the polymeric support according to the invention, said catalysts having structural formulae which can be further modified for controlling the catalytic activity of the catalyst.

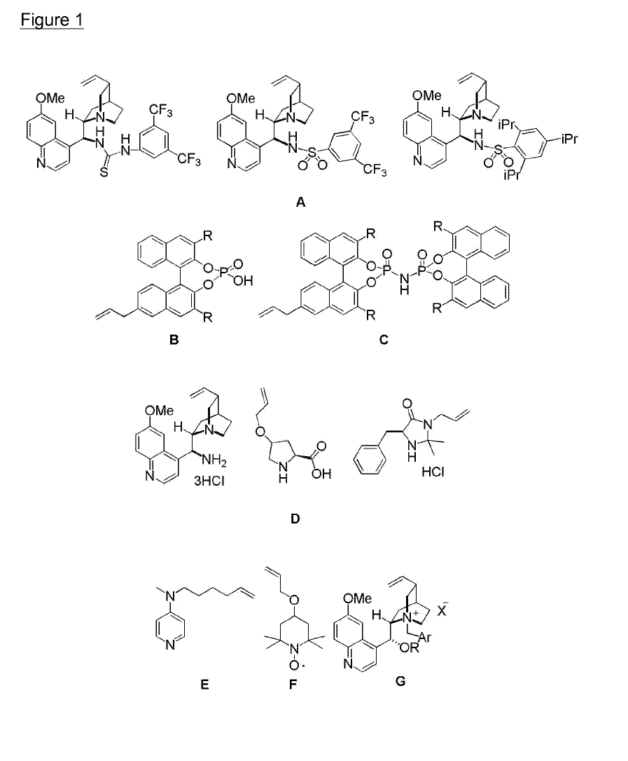

FIG. 2 shows the selectivity vs. the number of reaction cycles of one embodiment of the inventive material.

### Example 1

The photochemical immobilization of quinine and its derivatives is generally described in the following scheme.

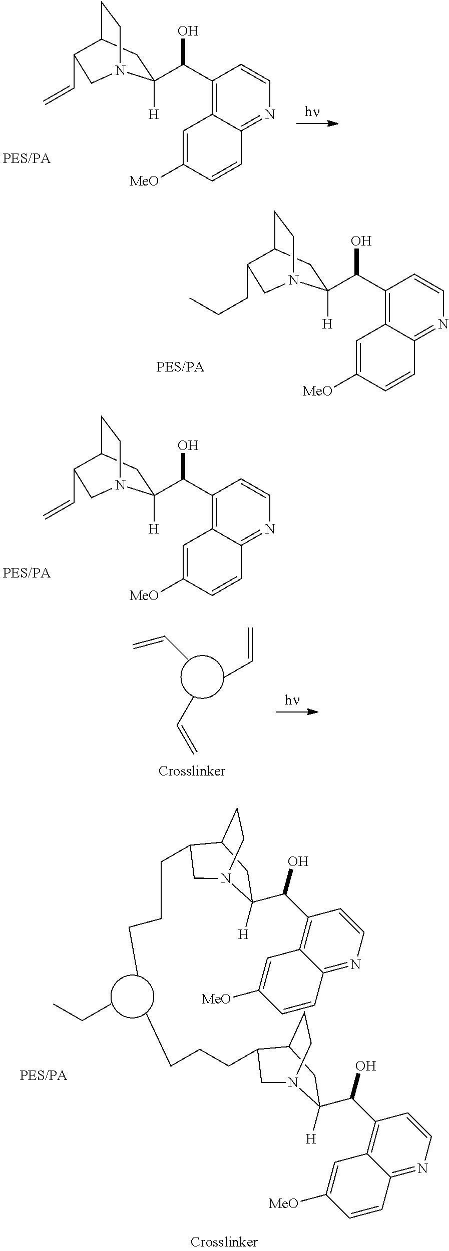

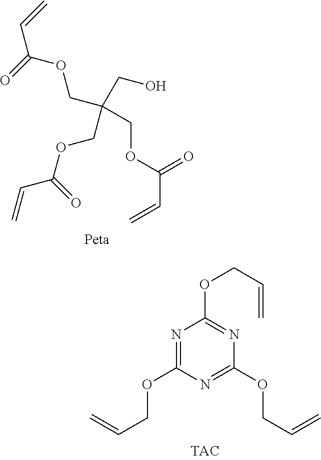 [SMILES: C=CC(=O)OCC(CO)(COC(=O)C=C)COC(=O)C=C ; C=CCOc1nc(OCC=C)nc(OCC=C)n1]

The catalytic activity and enantioselectivity are as follows:

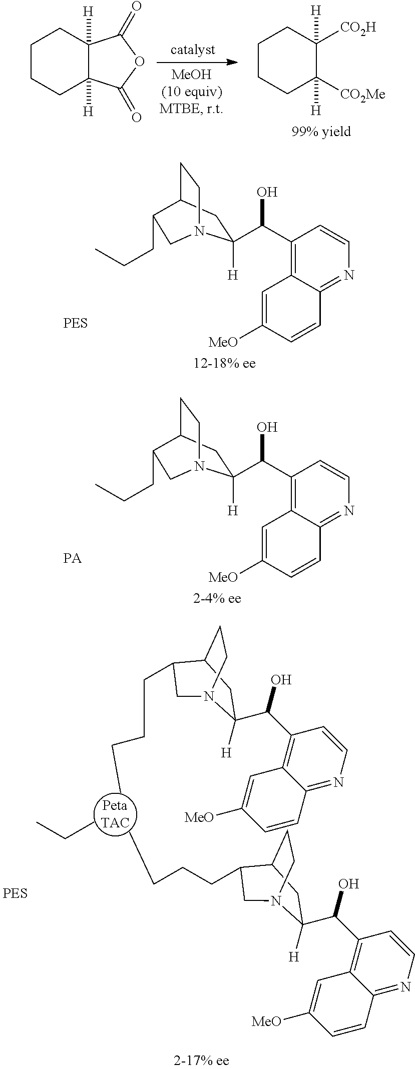

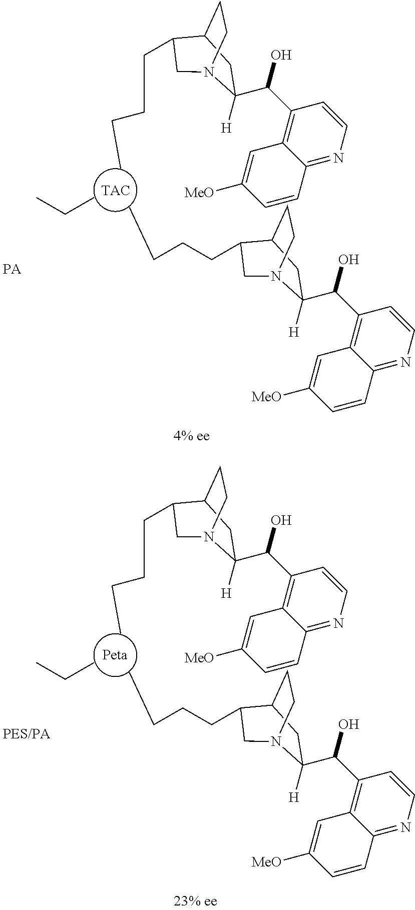

Recyclability of quinine-immobilized textile catalyst is as follows:

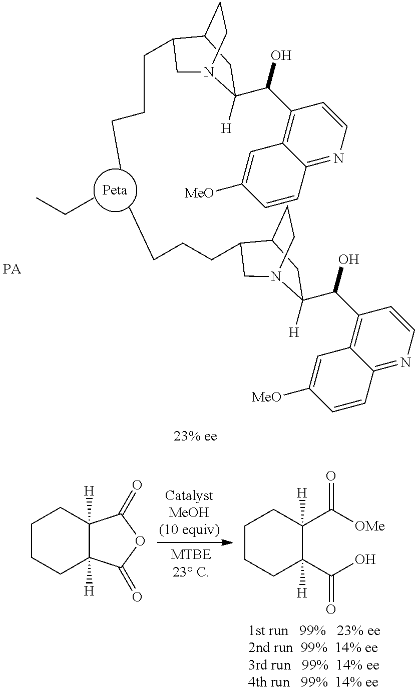

The quinine-immobilized textiles were used for desymmetrization of meso-anhydride and gave catalytic reactivity but enantioselectivities were inferior. However, through the recycling experiments, enantioselectivity was maintained even though ee values were low.

### Example 2

The photochemical immobilization of cinchona alkaloids and its derivatives is generally shown in the following schemes:

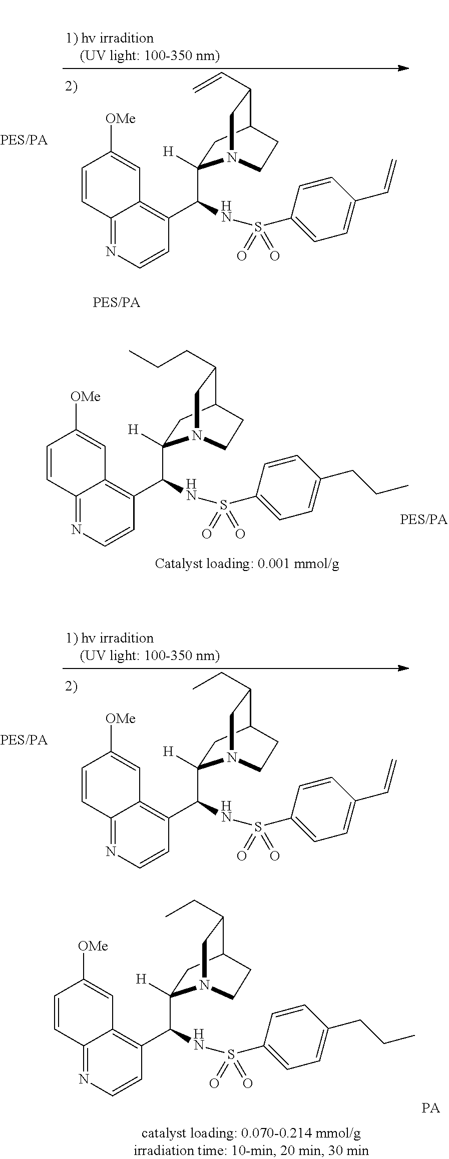 [SMILES: C=Cc1ccc(S(=O)(=O)N[C@@H](c2ccnc3ccc(OC)cc23)[C@@H]2CC3CC[N@]2CC3C=C)cc1 ; CCCc1ccc(S(=O)(=O)N[C@@H](c2ccnc3ccc(OC)cc23)[C@@H]2CC3CC[N@]2CC3CCC)cc1 ; C=Cc1ccc(S(=O)(=O)N[C@@H](c2ccnc3ccc(OC)cc23)[C@@H]2CC3CC[N@]2CC3CC)cc1 ; CCCc1ccc(S(=O)(=O)N[C@@H](c2ccnc3ccc(OC)cc23)[C@@H]2CC3CC[N@]2CC3CC)cc1]

| | | Concentration | |
|---|---|---|---|
| | Irradiation time | of monomer | Catalyst loading on textile |
| Entry | [min] | [mmol/g] | [mmol/g] |
| 1 | 10 | 0.05 | 0.166 |
| 2 | 10 | 0.1 | 0.188 |
| 3 | 20 | 0.1 | 0.214 |
| 4 | 30 | 0.1 | 0.194 |
| 5 | 30 | 0.25 | 0.070 |

### Example 3

The photochemical immobilization of a quinine-based sulfonamide catalyst on a polymeric support such PES/PA is generally shown in the following scheme:

The catalytic activity and enantioselectivity are as follows:

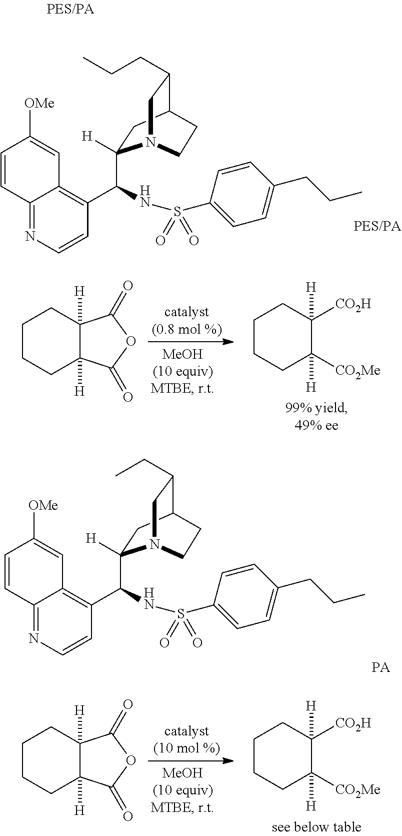

| | | | Catalyst | |
|---|---|---|---|---|
| | | | loading | |
| | Irradiation time | Concentration of monomer | on textile | ee |
| Entry | [min] | [mmol/g] | [mmol/g] | (%) |
| 1 | 10 | 0.05 | 0.166 | 13% |
| 2 | 10 | 0.1 | 0.188 | 25% |
| 3 | 20 | 0.1 | 0.214 | 38% |
| 4 | 30 | 0.1 | 0.194 | 32% |
| 5 | 30 | 0.25 | 0.070 | 54% |

### Example 4

The photochemical immobilization of a quinine-based sulfonamide catalyst on a support material or, more specifically, a textile material selected from woven or non-woven polyester, polyamide (or Nylon) and cotton is generally shown in the following scheme:

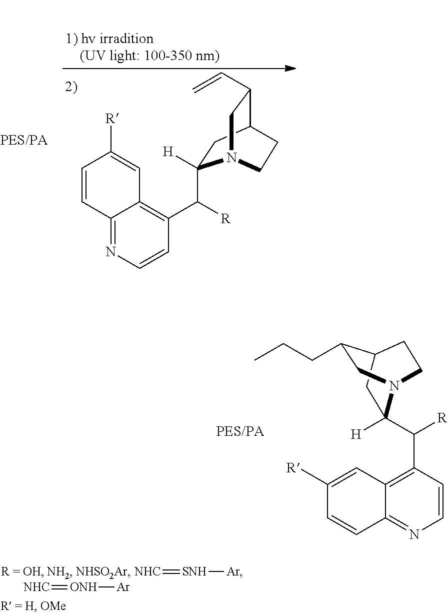

The photochemical immobilization of quinine-based sulfonamide catalysts on a polymeric support such as PA is generally shown for different loads in the following schemes:

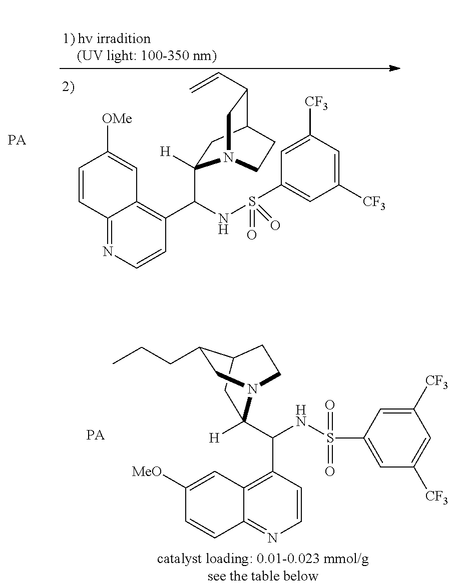

Concentration Irradiation Crosslinker Amount of

| | | | | | Catalyst |
|---|---|---|---|---|---|
| | | | | Amount of | loading |
| | Concentration | Irradiation | | crosslinker | on |
| Entry | of monomer | time | Crosslinker | mmol/g | textile |
| 1 | 0.025 | 2 × 5 min | | | 0.0122 |
| 2 | 0.1 | 2 × 5 min | | | 0.0154 |
| 3 | 0.3 | 2 × 5 min | peta | 0.17 | 0.0228 |
| 4 | 0.3 | 2 × 5 min | | | 0.0151 |
| 5 | 0.3 | 2 × 10 min | | | 0.0116 |

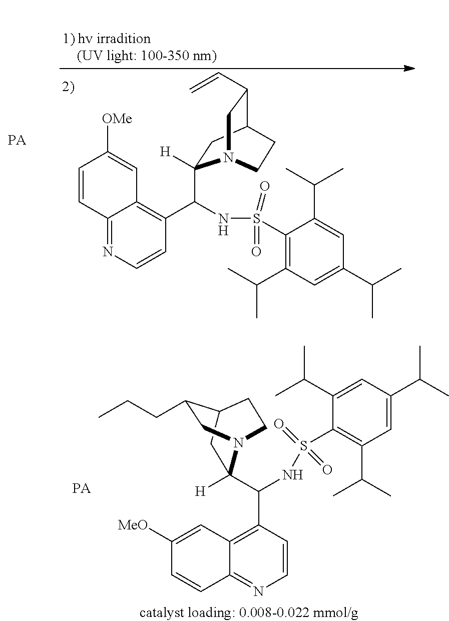

### Example 5

For a Quinine-thiourea catalyst on polyamide, the catalytic activity and enantioselectivity as well as recyclability of the textile catalyst have been evaluated as follows:

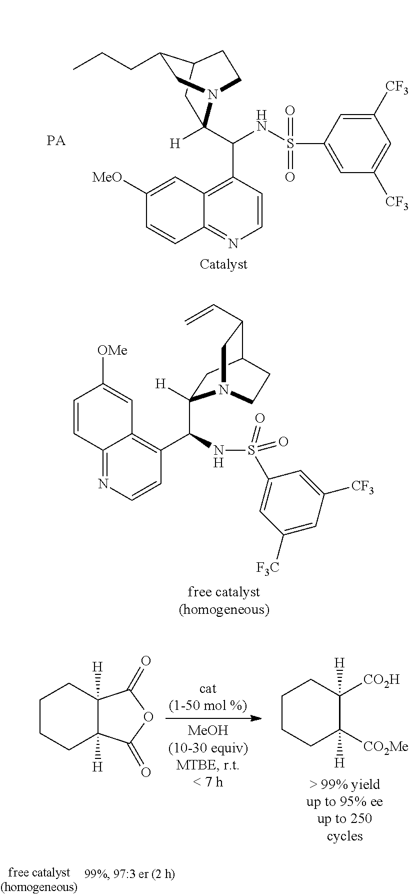

The catalytic reaction was conducted in 15 mL glass vial equipped with textile catalyst and magnetic stirring bar. Cyclohexanedicarboxylic anhydride was added and dissolved with MTBE (>10 mL). Then methanol (2-10 equiv) was added and the reaction mixture was stirred until the starting material consumed completely. Then the reaction vial was washed with MTBE (10 mL) 4 times and the combined organic layer was evaporated and dried under vacuo to afford the desired product (>99% purity by ¹H NMR) and then converted to the diastereomeric mixture to determine the ee values.

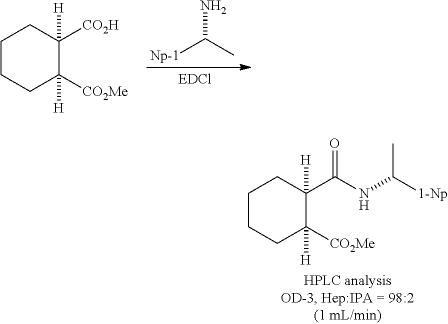

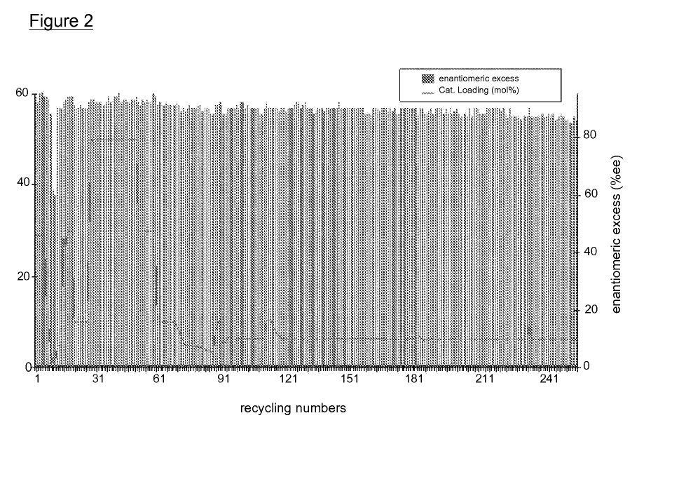

The recycling experiment was conducted using the same reaction vial by adding more substrates and solvent without further purification. The recyclability is shown in FIG. 2 showing that the inventive catalyst can be recycled for more than 100 times without a loss of activity.

The substrate scope of the immobilized QN-sulfonamide catalyst is shown in the following scheme:

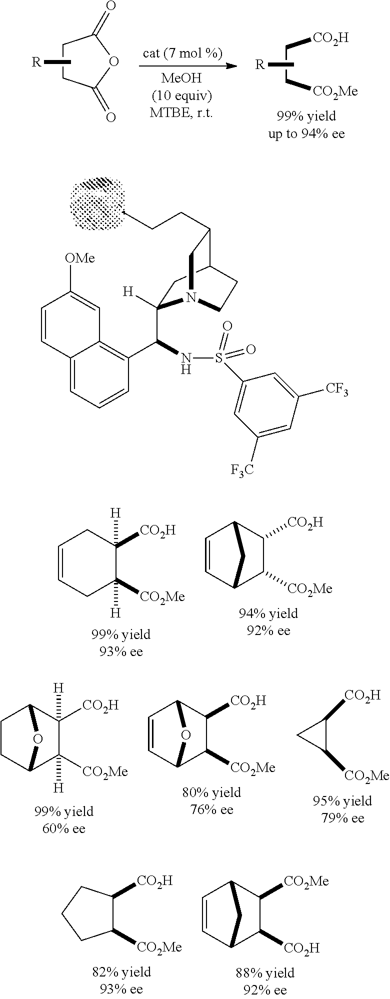

### Example 6

The immobilization of Quinine-thiourea catalyst on polyamide yielded catalysts with differing loadings 0.007 to 0.012 mmol/g support as follows:

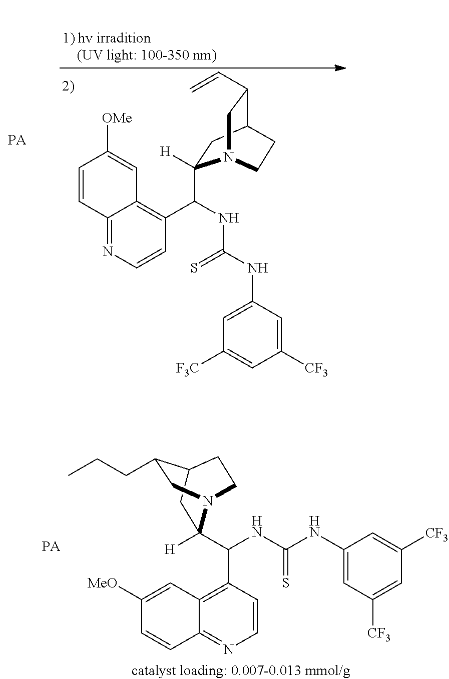

The application of quinine-thiourea textile catalyst revealed:

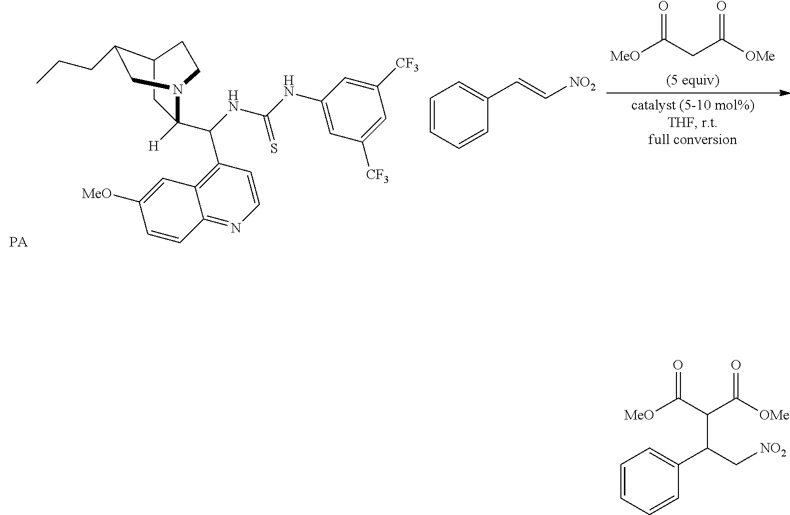

### Example 7

The synthesis and immobilization of phosphoric acid catalyst is exemplified for one compound in the following scheme:

A. Synthesis of allyl-substituted TRIP-phosphate

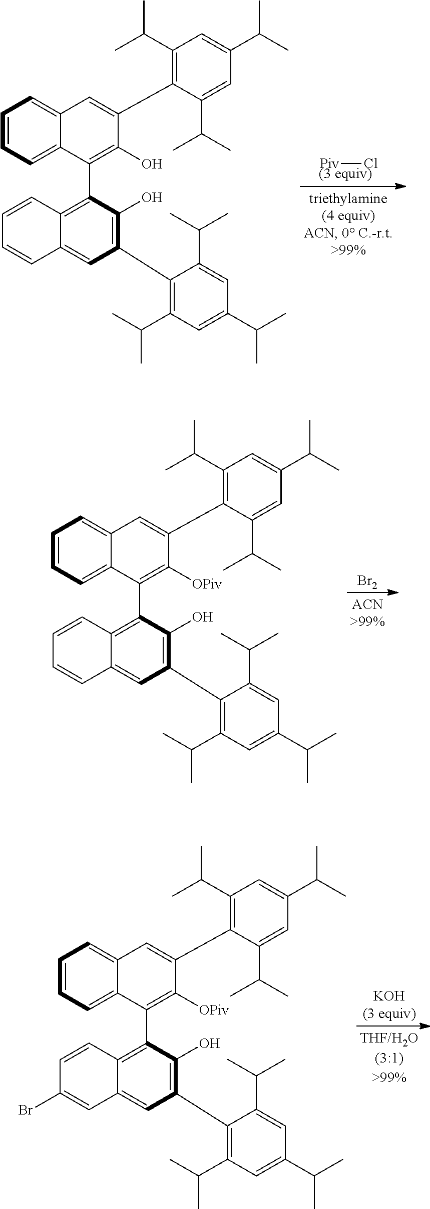

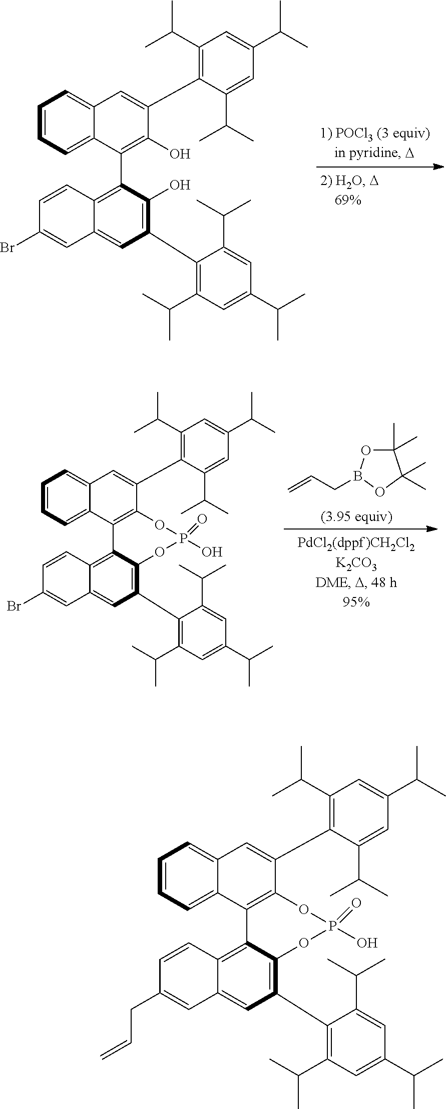

B. Immobilization of allyl substituted phosphoric acid

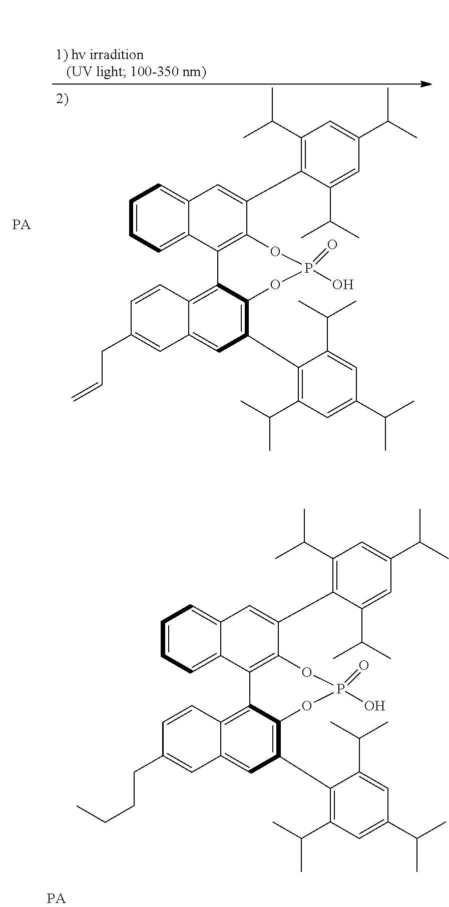

C. Comparison of a homogeneous catalyst and a heterogeneous catalyst

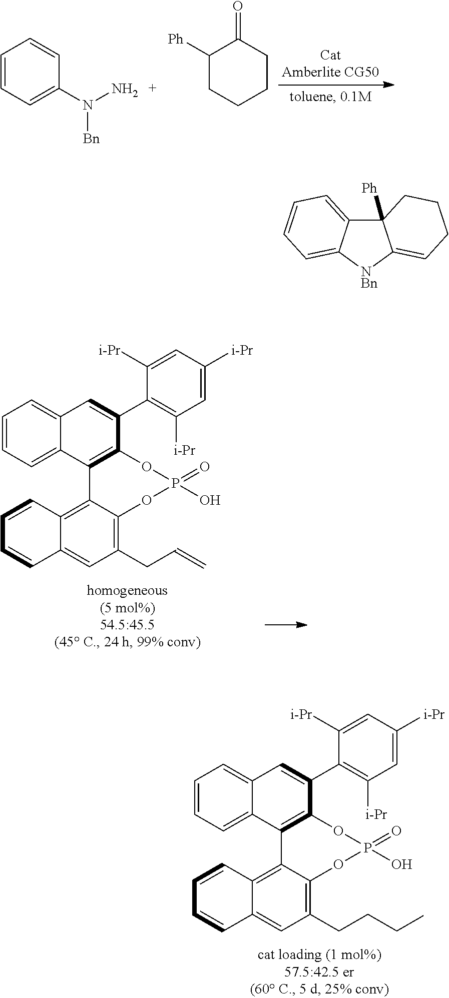

### Example 8

The synthesis and immobilization of imidodiphosphoric acid catalyst is exemplified for one compound in the following scheme:

A. Synthesis of allyl substituted imidodiphosphoric acid

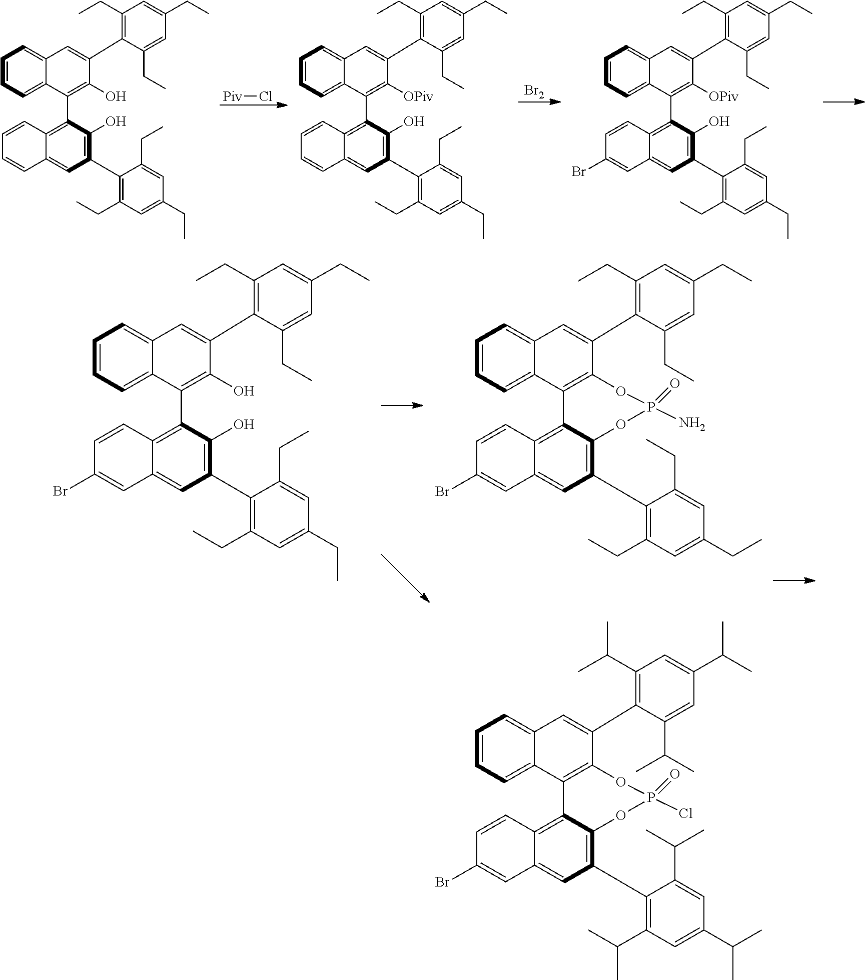

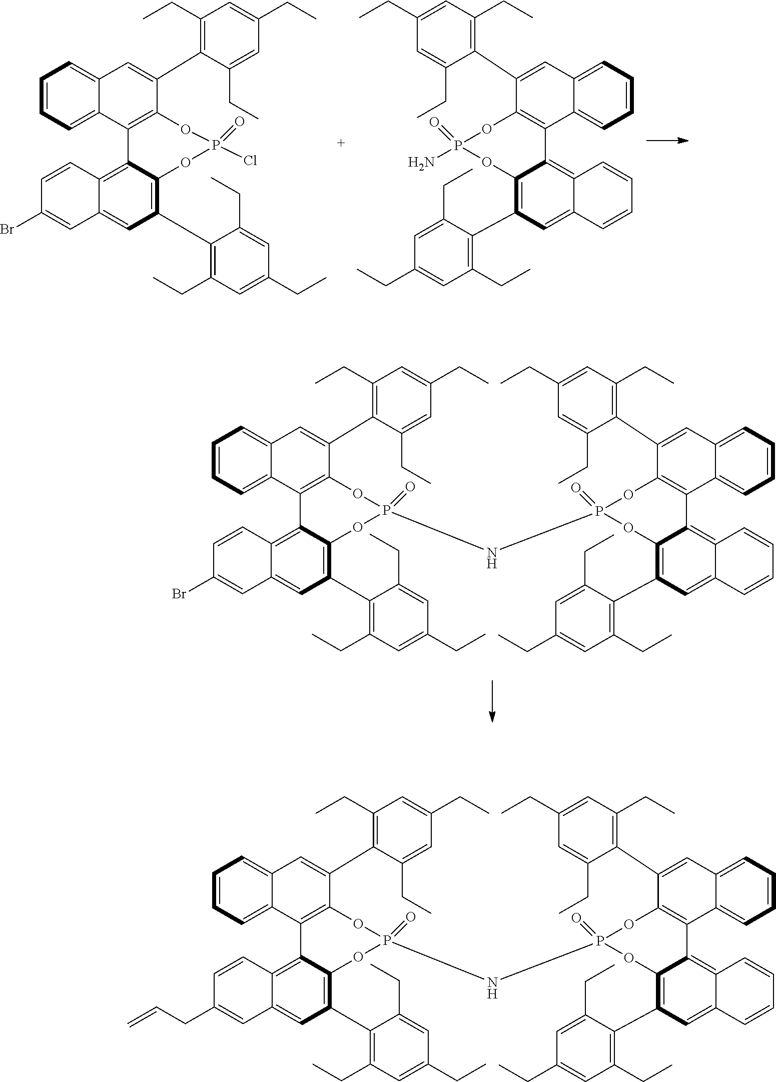

B. Immobilization of allyl substituted imidodiphosphoric acid

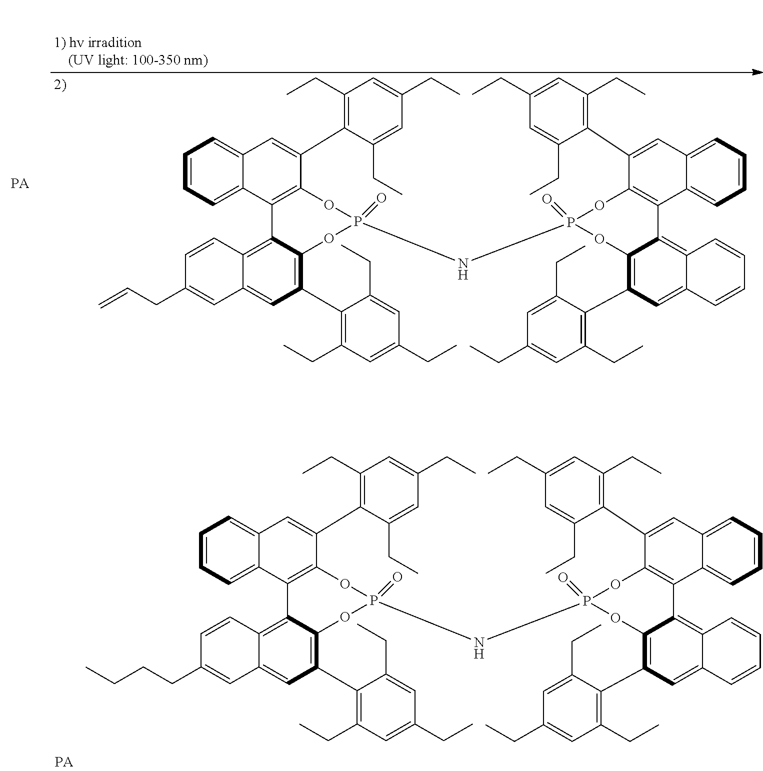

### Example 9

The synthesis and immobilization of primary- and secondary amine catalysts is exemplified for three compounds in the following schemes:

A. Synthesis and immobilization of Quinine-derived primary amine catalyst

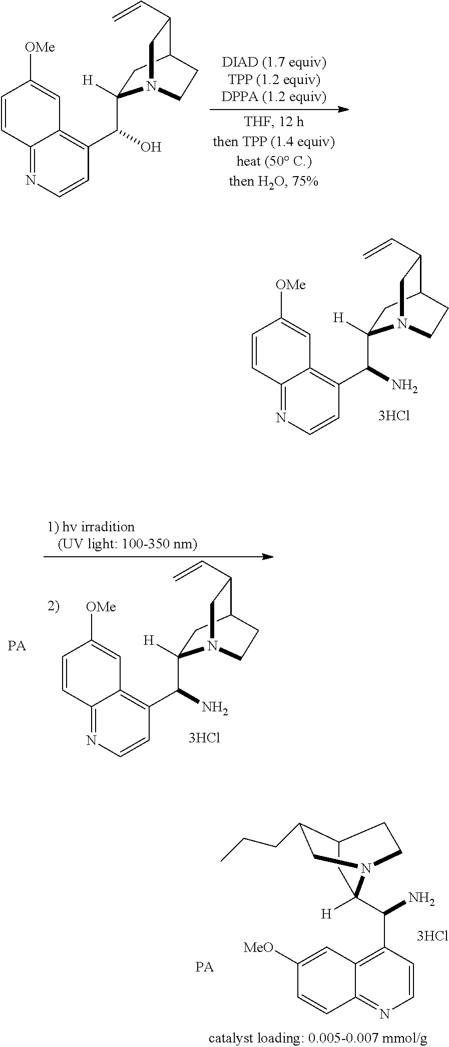 [SMILES: C=CC1C[N@@]2CCC1C[C@H]2[C@H](O)c1ccnc2ccc(OC)cc12 ; C=CC1C[N@@]2CCC1C[C@H]2[C@@H](N)c1ccnc2ccc(OC)cc12 ; C=CC1C[N@@]2CCC1C[C@H]2[C@@H](N)c1ccnc2ccc(OC)cc12 ; CCCC1C[N@@]2CCC1C[C@H]2[C@@H](N)c1ccnc2ccc(OC)cc12]

B. Synthesis and immobilization of proline catalyst and its use

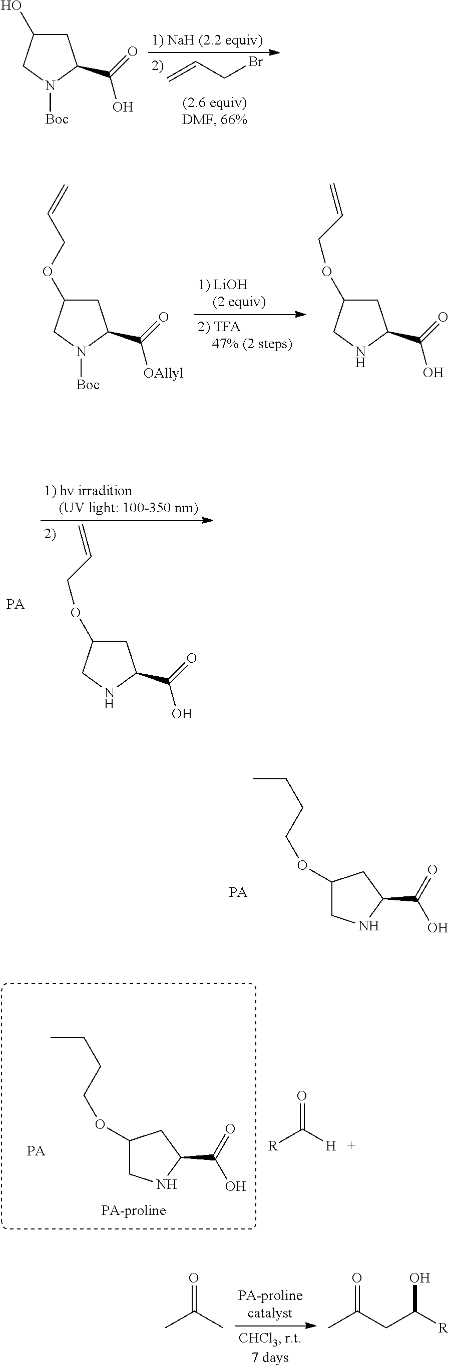

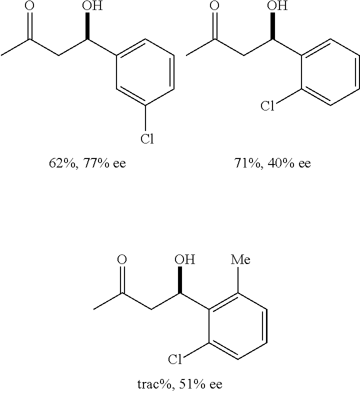

C. Synthesis and immobilization of phenylalanine-derived secondary amine

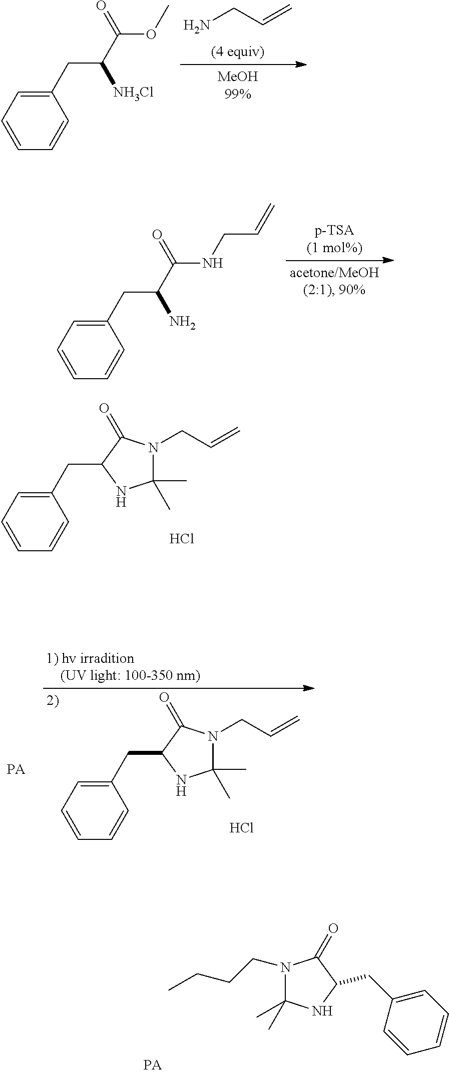

### Example 10

The synthesis and immobilization of a nucleophilic DMAP-type catalyst is exemplified for one compound in the following scheme:

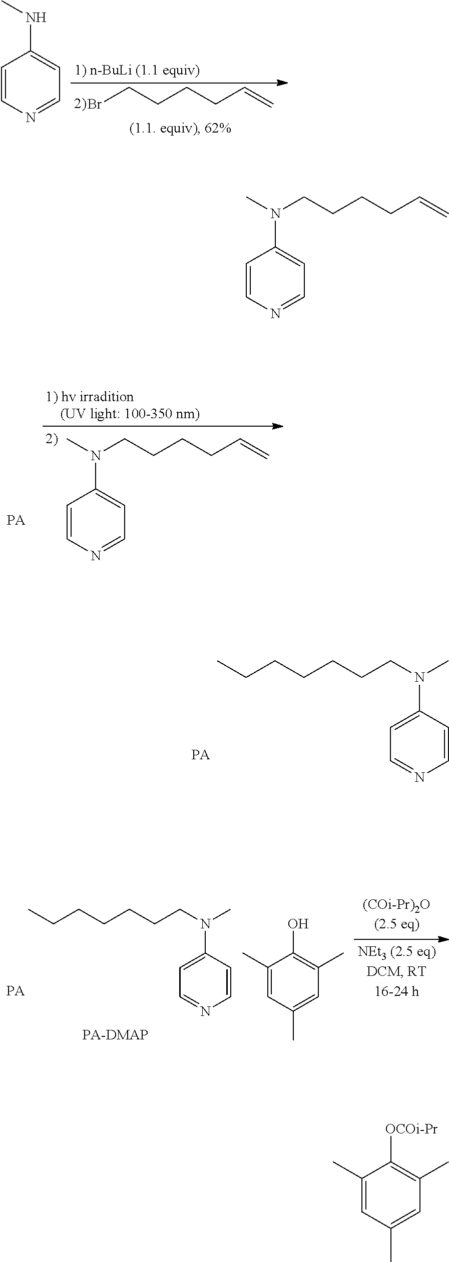

a. the number in the parenthesis are isolated yields of acylated product. b. after reaction, textile catalyst was washed with triethylamine to remove remained acidic by-product.

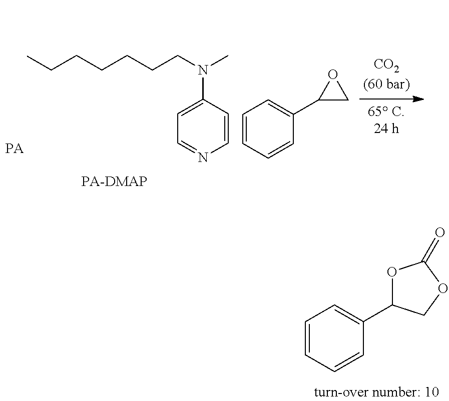

### Example 11

The synthesis and immobilization of a TEMPO-like catalyst is exemplified in the following scheme:

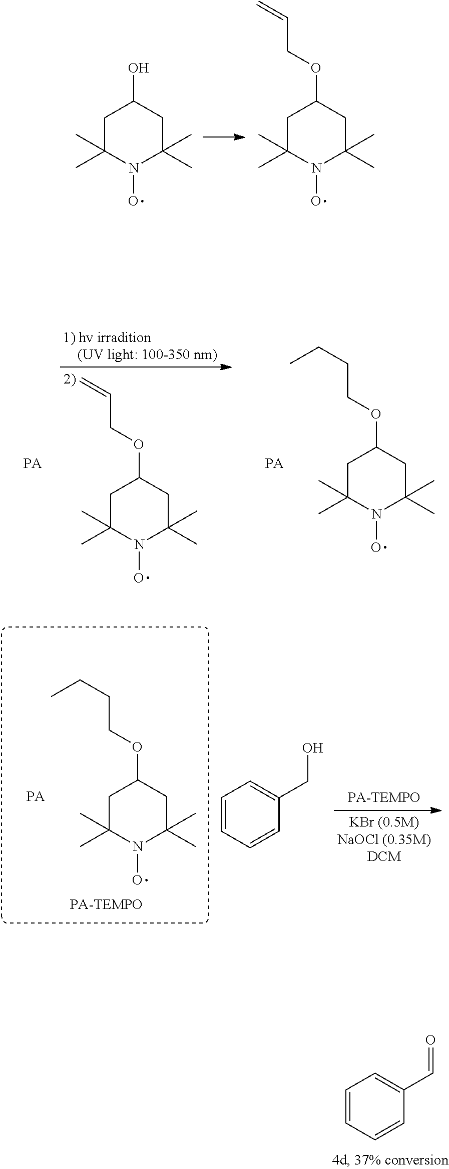

### Example 12

The synthesis and immobilization of a phase-transfer catalyst is exemplified in the following scheme

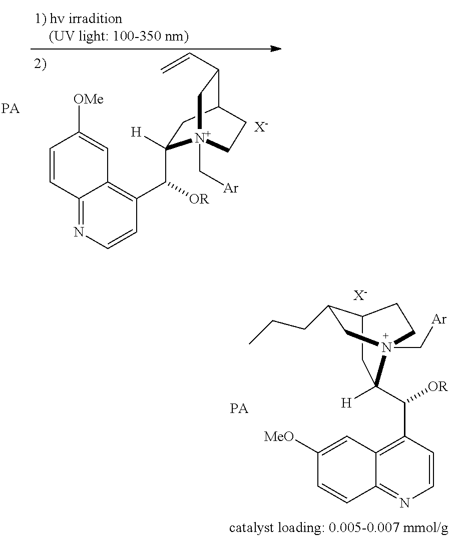

### Example 12

The synthesis and immobilization of a pTSA-like catalyst is exemplified in the following scheme:

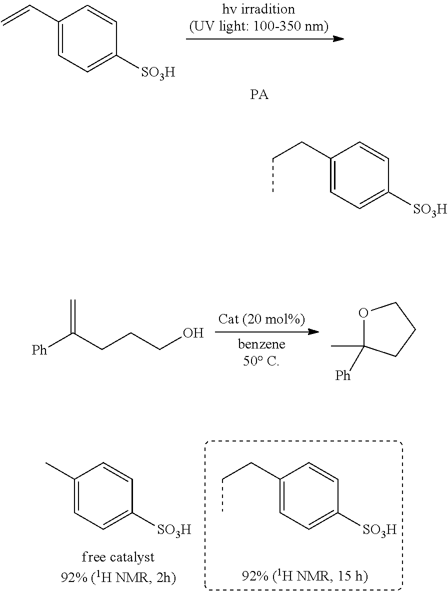

### Example 13

The immobilization of sulfonic acid catalysts using benzylic C—H activation are exemplified in the following scheme:

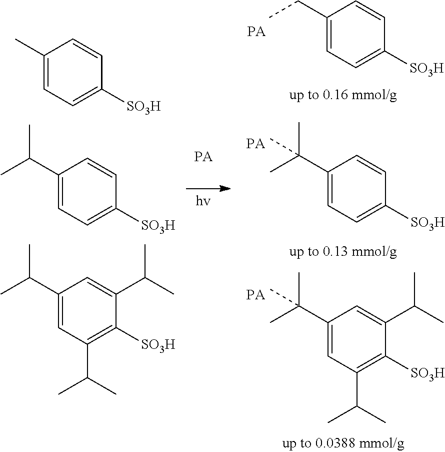

1.-13. (canceled)

14. A process for immobilizing an organic catalyst on a polymeric support, wherein said organic catalyst does not have any metal-centered catalytically active center, said process comprising covalently bonding the organic catalyst to the polymeric support by a photochemically induced reaction, wherein the polymeric support is selected from the group of natural or synthetic textile materials or mixtures thereof, selected from polyesters, polyamides, polyacrylates, polyolefins, cotton, rayon and wool.

15. The process according to claim 14, wherein the organic catalyst is an organic catalyst selected from any of cinchona alkaloid-based bifunctional catalysts, BINOL-based phosphoric acids, BINOL-based imidodiphosphoric acids, secondary- and primary amine catalysts, nitrogen-based nucleophilic catalysts, TEMPO as an organic oxidant, phase-transfer catalysts as well as imidodiphosphoric acids, which organic catalyst does not have any metal-centered catalytically active center.

16. The process according to claim 14, wherein the organic catalyst is an imidodiphosphoric acid of general formula (I): 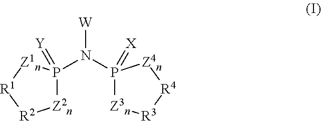
wherein:
X and Y may be, independently from each other, the same or different and represent O, S, Se and NR^{N},
Z¹ to Z⁴ may be, independently from each other, the same or different and represent O, S and NR^{N},
n stands for 0 or preferably 1,
W may be hydrogen, —OH, or halogen,
R¹ to R⁴ may be, independently from each other, the same or different and may be each an aliphatic, heteroaliphatic, aromatic or heteroaromatic group, each optionally being further substituted by one or more heterosubstituents, aliphatic, heteroaliphatic, aromatic or heteroaromatic groups whereby R¹ and R² form a ring system with Z¹ and Z² and R³ and R⁴ form a ring system with Z³ and Z⁴, respectively, and
R^{N} may be selected from hydrogen, C₁ to C₂₀ straight chain, branched chain or cyclic aliphatic hydrocarbons, optionally having one or more unsaturated bonds such as C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl or heterosubstituents, including its tautomeric and ionic forms, and derivatives thereof.

17. The process according to claim 16, wherein the organic catalyst is immobilized on the polymeric support via a photochemical reaction between at least one functional crosslinking olefinic group or benzylic group on the organic catalyst with the polymeric support, either directly or via a linker molecule having at least two vinylic or allylic olefinic groups.

18. The process of claim 17, wherein the functional crosslinking olefinic group is a vinyl or allyl group or benzyl group attached to the organic catalyst.

19. The process of claim 16, wherein the photochemical reaction is induced by irradiation with UV light.

20. The process of claim 19, wherein the irradiation with UV light is with UV light of a wavelength in the range of 100 to 350 nm.

21. The process of claim 20, wherein the irradiation with UV light is with UV light having a wavelength of 222 nm.

22. The process of claim 16, wherein the polymeric support is treated with a solution of the organic catalyst having at least one functional crosslinking olefinic group in a photochemically inert organic solvent, the obtained impregnated textile is irradiated with UV light and the obtained polymeric support having the organic catalyst covalently fixed thereon is recovered and optionally washed with an organic solvent.

23. Organic catalyst on a polymeric support, wherein said organic catalyst is covalently bonded to the polymeric support selected from the group of natural or synthetic textile materials or mixtures thereof, selected from polyesters, polyamides, polyacrylates, polyolefins, cotton, rayon and wool, and wherein the organic catalyst is a heterogeneous organic catalyst that does not have any metal-centered catalytically active center.

24. Organic catalyst on a polymeric support according to claim 23, wherein said organic catalyst is an organic catalyst covalently bonded to the polymeric support and wherein the polymeric support is selected from the group of natural or synthetic textile materials or mixtures thereof, selected from polyesters, polyamides, polyacrylates, polyolefins, cotton, rayon and wool and wherein the organic catalyst is selected from any of cinchona alkaloid-based bifunctional catalysts, BINOL-based phosphoric acids, BINOL-based imidodiphosphoric acids, secondary- and primary amine catalysts, nitrogen-based nucleophilic catalysts, TEMPO as an organic oxidant, phase-transfer catalysts as well as imidodiphosphoric acids.

25. Organic catalyst on a polymeric support according to claim 24, wherein the organic catalyst is an imidodiphosphoric acid of general formula (I): 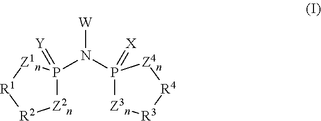
wherein:
X and Y may be, independently from each other, the same or different and represent O, S, Se and NR^{N},
Z¹ to Z⁴ may be, independently from each other, the same or different and represent O, S and NR^{N},
n stands for 0 or preferably 1,
W may be hydrogen, —OH, or halogen,
R¹ to R⁴ may be, independently from each other, the same or different and may be each an aliphatic, heteroaliphatic, aromatic or heteroaromatic group, each optionally being further substituted by one or more heterosubstituents, aliphatic, heteroaliphatic, aromatic or heteroaromatic groups whereby R¹ and R² form a ring system with Z¹ and Z² and R³ and R⁴ form a ring system with Z³ and Z⁴, respectively, and
R^{N} may be selected from hydrogen, C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl, each hydrocarbon optionally being substituted by one or more groups selected from C₁ to C₂₀ straight chain, branched chain or cyclic aliphatic hydrocarbons, optionally having one or more unsaturated bonds such as C₁-C₂₀-alkyl, C₂-C₂₀-alkenyl or C₂-C₂₀-alkinyl, C₃-C₈-heterocycloalkyl or C₆ to C₂₀ aromatic hydrocarbon and partially arene-hydrogenated forms such as aryl, aryl-(C₁-C₆)-alkyl, heteroaryl-(C₁-C₆)-alkyl or heterosubstituents, including its tautomeric and ionic forms, and derivatives thereof.

26. Organic catalyst on a polymeric support being obtainable by a process according to claim 14.

27. A process comprising catalyzing a catalytically promoted chemical reaction with a catalyst, wherein the catalyst is an organic catalyst on a polymeric support according to claim 22.
